(19) United States
(12) Patent Application Publication
(10) Pub. No.: US 2004/0083835 A1
(43) Pub. Date: May 6, 2004

(54) Insertion tube methods and apparatus

(76) Inventors: William L. Casper, Rigby, ID (US); Don T. Clark, Idaho Falls, ID (US); Blair K. Grover, Idaho Falls, ID (US); Rodney O. Mathewson, Idaho Falls, ID (US); Craig A. Seymour, Idaho Falls, ID (US)

Correspondence Address: Alan D. Kirsch, P.O. Box 1625, Idaho Falls, ID, 83415-3899, US

(21) Appl. No.: 10/285,786

(22) Filed: Oct. 31, 2002

(51) Int. Cl. G01N 33/00; E21B 49/00

(52) U.S. Cl. 73/866.5; 73/866

(57) ABSTRACT

An apparatus which facilitates placing an instrumented probe into a media, including a plurality of probe casings having first and second ends, the first end of one probe casing being configured to selectively couple with the second end of another probe casing at a casing joint to form an insertion tube, the insertion tube having an instrument receiving end, a surface end, and an insertion tube wall which together define a central cavity, and wherein the casing joint includes a seal which functions as a substantial barrier to contaminants.

### GOVERNMENT RIGHTS

This invention was made with Government support under Contract DE-AC07-99ID13727 awarded by the U.S. Department of Energy. The Government has certain rights in the invention.

### TECHNICAL FIELD

The invention relates to methods and apparatus for subsurface testing. More specifically the invention relates to methods and apparatus for placing instrumented probes into the ground.

### BACKGROUND OF THE INVENTION

Water and associated contaminants seep into the ground and travel through a subsurface region known as the vadose zone (a region of unsaturated soil). How the water and associated contaminants move in the vadose zone, to a large degree, determines how much contamination (such as gasoline additives, agricultural chemicals, or buried waste leakage) may end up in a water supply (such as an aquifier). Therefore, gaining an understanding of how the water and associated contaminants move in the vadose zone is valuable for appropriate waste containment. Information regarding the movement of water and associated contaminants in the vadose zone is generally acquired through the use of subsurface probes or similar testing devices. Several apparatus and methods have been used to facilitate such testing and information gathering. Some of these apparatus and methods involve obtaining samples of subsurface liquids, while others test soil moisture or other parameters.

Monitoring and testing to determine the movement of subsurface water and associated contaminants is particularly valuable when dealing with waste disposal sites that contain radiological contaminants or other hazards. However, as described above, placing probes into the subsurface for data collection in such sites has not been feasible, because the placing of such probes would require drilling or coring which would bring contaminated “cuttings” to the surface and would create a pathway through which contaminated emissions may escape. As a result, testing probes have typically been placed in areas around such waste sites. Unfortunately, such probe placement only provides information when the contaminants have already migrated outside of the waste disposal site area. Moreover, at the point when the contaminants have already migrated outside of the waste disposal site area, it is likely that a major contaminant plume already exists in the subsurface soil and aquifer making remediation and containment efforts much more difficult and costly.

In view of the foregoing, it would be highly desirable to provide methods and apparatus which facilitate the installation of subsurface testing instruments in both contaminated and non-contaminated areas, while substantially avoiding these and other shortcomings of prior devices.

### BRIEF DESCRIPTION OF THE DRAWINGS

Preferred embodiments of the invention are described below with reference to the following accompanying drawings.

FIG. 1 is a front elevational view, partly in section, showing two probe casings in accordance with one embodiment of the present invention.

FIG. 2 is a front elevational view, partly in section, showing the probe casings of FIG. 1 and one possible instrumented probe positioned for use in a substrate.

FIG. 3 is a front elevational view, partly in section, showing the probe casings of FIG. 1 and another possible instrumented probe positioned for use in a substrate.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

The invention relates to methods and apparatus for subsurface testing. More specifically, the invention relates to methods and apparatus for placing instrumented probes into a substrate. The invention allows such placement to be carried out in either contaminated or non-contaminated sites without the need for drilling or coring. In one implementation, the method includes placing an instrumented probe into the substrate using direct push, sonic drilling, or a combination of direct push and sonic drilling.

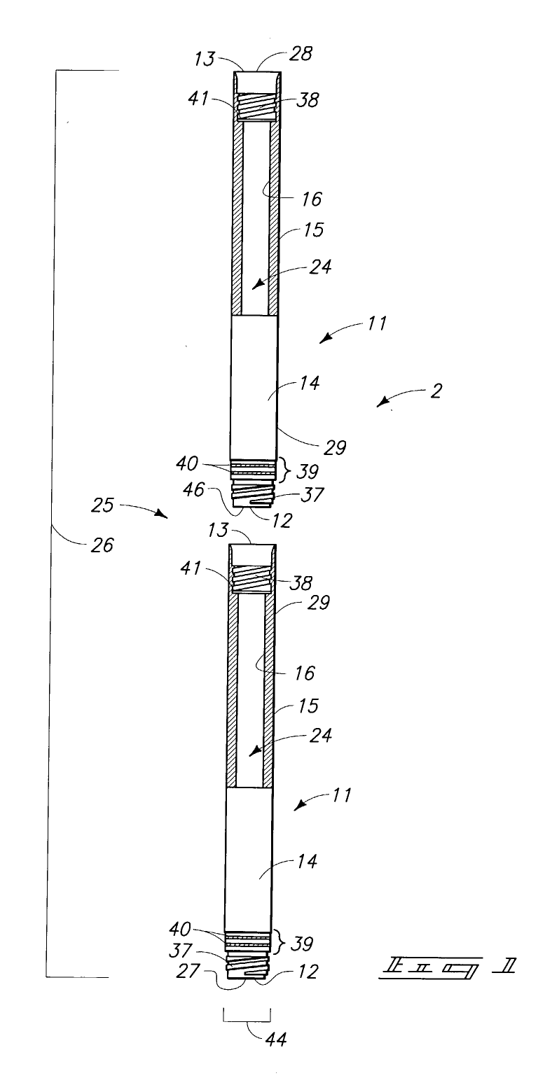

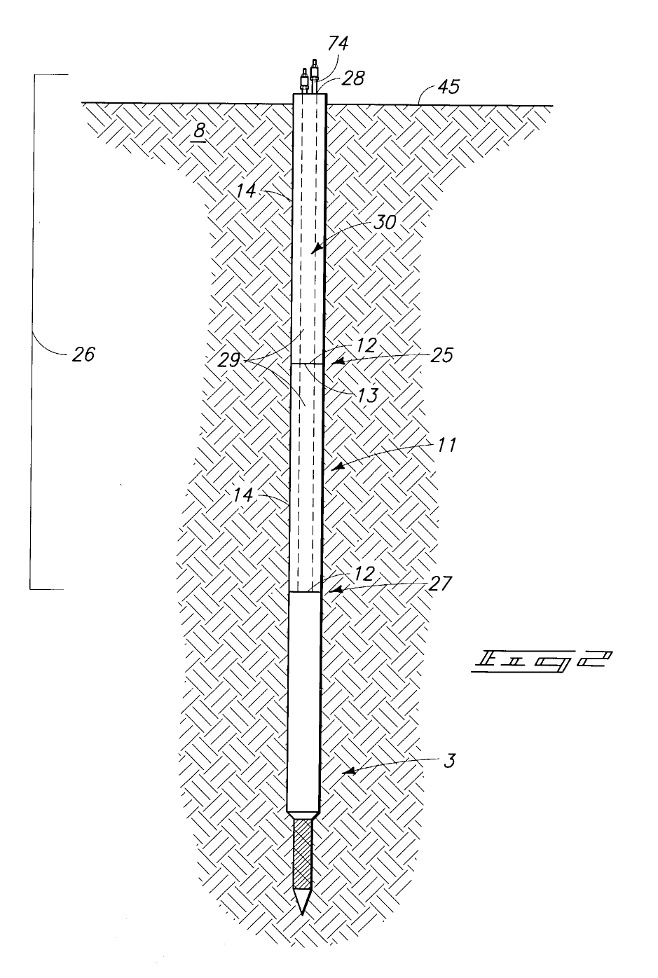

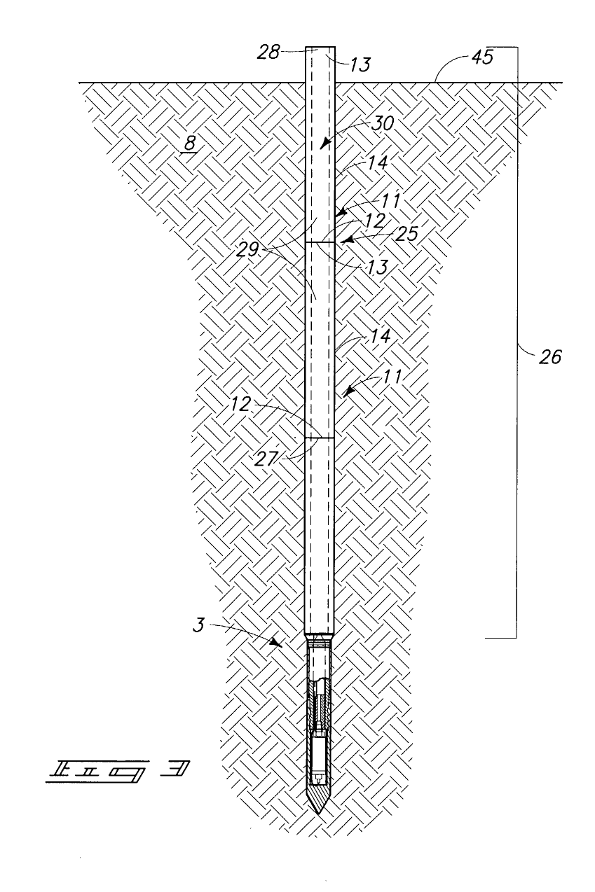

Shown in the various drawings is an apparatus 2 which facilitates placing an instrumented probe into a sample or the earth (hereinafter “the ground”) 8 (FIGS. 2 and 3). The apparatus 2 may be used to facilitate the placement of a variety of instrumented probes 3 into the ground 8, as will be described in detail below with reference to FIGS. 1-3.

The apparatus 2 may include one or more probe casings or insertion tubes 11. For ease of discussion, FIG. 1 depicts two such probe casings 11. Each of these probe casings 11 has an end 12 and an end 13 which are open. A sidewall 14 extends between the open ends 12 and 13. The sidewall 14 has an outer surface 15 and an inner surface 16. A probe casing cavity 24 is defined by the ends 12 and 13, and sidewall 14 of the probe casing 11. In the illustrated embodiment, the inner surface 16 is an inner cylindrical surface and the probe casing cavity 24 is a generally cylindrical void which runs the length of the probe casing 11; however other shapes are possible. In FIG. 1, a portion of the sidewall 14 has been removed, so that the probe casing cavity 24 may be seen.

As shown in the various Figures, the end 12 of one probe casing 11 is configured to be selectively coupled with the end 13 of another probe casing 11 at a casing joint 25 to form an insertion tube 26, as the instrumented probe 3 is driven into the ground 8. In the context of this document, the term “insertion tube” 26 is defined to mean a plurality of probe casings 11 which have been coupled, or a plurality of probe casings 11 which are configured to be selectively coupled.

The insertion tube 26 which is formed from the selectively coupled probe casings 11 includes an instrument receiving end 27, a surface end 28, and an insertion tube wall 29 which together define a central cavity 30 (indicated by phantom lines in FIGS. 2 and 3). The probe casing cavities 24 of each of the probe casings 11 which have been selectively coupled to form the insertion tube 26, together define the central cavity 30 of the insertion tube 26. The central cavity 30 is a generally cylindrical void which runs the length of the insertion tube 26; however, other shapes are possible.

As described above, the individual probe casings 11 are selectively coupled to form the insertion tube 26. The probe casings 11 may be selectively coupled using any suitable arrangement. In the embodiments depicted in FIGS. 1-3, the probe casings 11 have male and female threaded ends 37 and 38 which are used to selectively couple the respective probe casings 11. Specifically, the male threaded end 37 of one probe casing 11 is configured to selectively couple with the female threaded end 38 of another probe casing 11 at a casing joint 25 to form the insertion tube 26. The casing joints 25 respectively include a seal 39 which functions as a substantial barrier to contaminants. The seal 39 functions to substantially prevent contaminants outside of the insertion tube 26 from moving through the casing joint 25 and into the central cavity 30 of the insertion tube 26. Similarly, the seal 39 also functions to substantially prevent any contaminants which are located within the central cavity 30 from moving through the casing joint 25 and outside of the insertion tube 26.

In the embodiment shown in FIG. 1, the seal 39 comprises a plurality of seal members. Specifically, in the depicted embodiment, the seal 39 has two o-ring seals 40 which function as a substantial barrier to contaminants. The probe casings 11 also include bearing surfaces 41 and 46 which function to isolate the seal 39 and to protect the seal 39 from large loads while the insertion tube 11 is being used to insert an instrumented probe 3 into the ground (see FIGS. 2 and 3).

In the embodiment of FIG. 1, the probe casings 11 are stainless steel. However, any suitable material may be utilized to construct the probe casings 11. The outer wall or sidewall 14 of the probe casings 11 define an outside diameter 44. In one embodiment, the outside diameter 44 is less than 5⅝ inches. In the depicted embodiment, the outside diameter 44 is about two and one-half inches, and the thickness of the outer wall 14 is about 0.25 inches thick; other sizes are employed in alternative embodiments. The length of the probe casings 11 can be varied to suit various needs. In the illustrated embodiment, the probe casings 11 are of a size and weight that allow the probe casings 11 to be assembled by hand in the field to form the insertion tube 26 as the instrumented probe is being driven into the ground 8.

As shown in FIG. 1, the male and female threaded ends 37 and 38 are configured so that the male threaded end 37 of one probe casing 11 and the female threaded end 38 of another probe casing 11 may be easily coupled. In one embodiment, selectively coupling the male threaded end 37 of one probe casing 11 and the female threaded end 38 of another probe casing 11 requires less than four turns to fully engage the casing joint 25 and the seal 39. More particularly in the depicted embodiment, selectively coupling the male threaded end 37 of one probe casing 11 and the female threaded end 38 of another probe casing 11 requires about two and one-half turns to fully engage the casing joint 25 and the seal 39. The advantage of this is to ensure that wiring or tubing (for example, extending from an attached instrument) is minimally twisted. This thread configuration also facilitates easy assembly and disassembly of the insertion tube 26 in the field. The insertion tube 26 so formed is of an adequate durability to facilitate installation of an instrumented probe 3 into a ground 8 by direct push, by sonic drilling, or by a combination of direct push and sonic drilling.

In one embodiment, a first probe casing 11 is selectively coupled with an instrumented probe 3, as is described in detail below. After selectively coupling the first probe casing 11 with the instrumented probe 3, the instrumented probe 3 and at least a portion of the coupled first probe casing 11 are inserted into the ground 8 by direct push, by sonic drilling, or by a combination of direct push and sonic drilling. Then additional probe casings 11 are selectively coupled (one at a time), in series, to the first probe casing 11 to form an insertion tube 26 as the instrumented probe 3 is driven progressively deeper into the ground 8. The seal 39 at each of the casing joints 25 functions as a substantial barrier to contaminants, thereby preventing contaminants in the ground 8 from passing through a casing joint and entering the central cavity 30 of the insertion tube 26. Therefore, the insertion tube 26 facilitates placing an instrumented probe 3 into the ground 8 without the need for prior excavation or drilling. Examples of such instruments and probes include suction lysimeters and tensiometers. The apparatus 2 can also be used with other instrument types used for subsurface testing.

In operation, an instrumented probe 3 is selectively coupled to the instrument receiving end 27 of the insertion tube 26 (engaging a seal 39 therebetween), and is driven into the ground 8 as described above. After the final probe casing 11 has been added to the insertion tube 26, the surface end 28 of the insertion tube 26 typically protrudes from the surface 45 of the ground 8 (FIGS. 2 and 3). The central cavity 30 of the insertion tube 26 is configured to pass at least one instrument conduit 74 (FIG. 2) which extends from the instrumented probe 3 to the land's surface 45. In operation, the instrument conduits which are received by the central cavity 30, may function to transfer a liquid, to transfer a gas, to transfer data, and/or any combination of such.

FIGS. 1-3 also depict methods of forming an insertion tube 26 for placement of an instrumented probe 3 into a ground 8. One method includes providing a plurality of probe casings 11 which are to be used to form an insertion tube 26. The male threaded end 37 of a first probe casing 11 is configured to selectively couple with the female threaded end 38 of a second probe casing 11 at a casing joint 25 to form an insertion tube 26. At least one seal 39 is provided at the casing joint 25 where the male and female threaded ends 37 and 38 are to be selectively coupled. The first and second probe casings 11 are then turned relative to each other to selectively couple the male threaded end 37 of the first probe casing 11 with the female threaded end 38 of the second probe casing 11 to form the insertion tube 26. In one embodiment, the first and second probe casings 11 are turned less than four turns relative to each other to fully engage the casing joint 25 and the seal 39. In one embodiment, the first and second probe casings 11 are turned about two and one-half turns relative to each other to fully engage the casing joint 25 and the seal 39. The casing joints do not gall or friction weld to one another, and the joint between the lowermost casing and the instrumented probe does not gall or friction weld together in view of the thread arrangement. The components can be readily removed from one another.

As one possible example, the casings of the respective instrumented probes 3 of FIGS. 2 and 3 comprise or are defined by stainless steel. However, any suitable material may be used to construct the casings. In one embodiment, the casing comprises stainless steel, and is of adequate durability for installation into a substrate by direct push, by sonic drilling, or by a combination of direct push and sonic drilling. When the probe casings are in the media after advancing an instrument into the media, they may be pressure tested from the top.

The invention provides robust insertion tubes that are particularly useful for driving into highly contaminated waste, as well as other uses. The insertion tubes can be driven into difficult materials (e.g., hardened soils, concrete, steel, other metals, etc.) that would typically damage other tools. In the illustrated embodiments, small diameter designs are employed that require less energy for installation into a sample. Reduced energy requirements allow for smaller driving equipment resulting in lower cost.

In one embodiment, the probe casing is of all stainless steel construction for maximum corrosion resistance and long term usage. A double (redundant) o-ring seal on a non-load bearing surface impedes contamination transfer from the sample (e.g., the soil) to ground surface. The redundant seal impedes contaminants or toxic materials from interfering with or damaging instrument probes. A robust design has been disclosed for direct push, sonic, and combined direct-push and sonic loading. The design supports structural integrity and the ability to transport delicate instrumentation without damage, to a desired ground depth. A thread configuration has been disclosed that allows for assembly with minimal rotation while maintaining structural integrity, to prevent damage to instrumentation (electrical leads, tubing, etc.) as well as for field handling ease. In one embodiment, a small diameter size is used with a light casing segment for handing ease in the field. The probe casing is structurally durable and designed for retraction, replacement, and/or reuse at other sites. The casing joints do not gall or friction weld to one another, and the joint between the lowermost casing and the instrumented probe does not gall or friction weld together in view of the thread arrangement. When the probe casings are in the media after advancing an instrument into the media, they may be pressure tested from the top.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

1. A method of placing an instrumented probe into a media, comprising:
providing an instrumented probe which is to be driven into a media, the instrumented probe having a tip portion and a drive portion;
providing a plurality of probe casings which may be selectively coupled to form an insertion tube as the instrumented probe is progressively driven deeper into the media;
coupling a first probe casing to the drive portion of the instrumented probe;
driving at least a portion of the instrumented probe and the coupled first probe casing into the media; and
after the driving at least a portion of the instrumented probe and the coupled first probe casing into the media, selectively coupling additional probe casings to form an insertion tube as the instrumented probe is driven progressively deeper into the media.

2. An apparatus which facilitates placing an instrumented probe into a media, comprising:
a plurality of probe casings having first and second ends, the first end of one probe casing being configured to selectively couple with the second end of another probe casing at a casing joint to form an insertion tube, the insertion tube having an instrument receiving end, a surface end, and an insertion tube wall which together define a central cavity, and wherein the casing joint includes a seal which functions as a substantial barrier to contaminants.

3. The apparatus of claim 2, wherein the seal substantially prevents contaminants outside the insertion tube from moving through the casing joint and into the central cavity of the insertion tube.

4. The apparatus of claim 2, wherein the seal substantially prevents contaminants which are located within the central cavity of the insertion tube from moving through the casing joint and outside of the insertion tube.

5. The apparatus of claim 2, wherein the seal comprises a plurality of seals.

6. The apparatus of claim 2, wherein the seal comprises two seals.

7. The apparatus of claim 2, wherein bearing surfaces isolate the seal from large loads.

8. An apparatus which facilitates placing an instrumented probe into a media, comprising:
a plurality of probe casings having male and female threaded ends, the male threaded end of one probe casing being configured to selectively couple with the female threaded end of another probe casing at a casing joint to form an insertion tube, the insertion tube having an instrument receiving end, a surface end, and an insertion tube wall which together define a central cavity, and wherein the casing joint includes a seal which functions as a substantial barrier to contaminants.

9. The apparatus of claim 8, wherein the seal comprises a plurality of seals.

10. The apparatus of claim 8, wherein the seal comprises two seals.

11. The apparatus of claim 8, wherein bearing surfaces isolate the seal from large loads.

12. The apparatus of claim 8, wherein the seal substantially prevents contaminants outside the insertion tube from moving through the casing joint and into the central cavity of the insertion tube.

13. The apparatus of claim 8, wherein the seal substantially prevents contaminants which are located within the central cavity of the insertion tube from moving through the casing joint and outside the insertion tube.

14. The apparatus of claim 8, wherein selectively coupling the male threaded end of one probe casing with the female threaded end of another probe casing requires less than four turns to fully engage the casing joint and the seal.

15. The apparatus of claim 8, wherein selectively coupling the male threaded end of one probe casing with the female threaded end of another probe casing, requires about two and one-half turns to fully engage the casing joint and the seal.

16. The apparatus of claim 8, wherein the probe casings have an outer wall defining an outside diameter, and wherein the outside diameter is less than five and five-eighths inches.

17. The apparatus of claim 8, wherein the probe casings have an outer wall, and wherein the thickness of the outer wall is about 0.25 inches thick.

18. The apparatus of claim 8, wherein the probe casings are comprised of stainless steel.

19. The apparatus of claim 8, wherein the probe casings are comprised of corrosion resistant material.

20. The apparatus of claim 8, wherein the probe casings may be assembled by hand in the field to form the insertion tube.

21. The apparatus of claim 8, wherein the central cavity of the insertion tube is configured to in operation receive at least one instrument conduit.

22. The apparatus of claim 21, and further comprising an instrument conduit in the central cavity wherein the at least one instrument conduit is a liquid transfer conduit.

23. The apparatus of claim 21, and further comprising at least one instrument conduit in the central cavity wherein the at least one instrument conduit is a gas transfer conduit.

24. The apparatus of claim 21, and further comprising at least one instrument conduit in the central cavity wherein the at least one instrument conduit is a data transfer cable.

25. The apparatus of claim 8, wherein the insertion tube is of adequate durability to facilitate installation of an instrumented probe into a media by direct push.

26. The apparatus of claim 8, wherein the insertion tube is of adequate durability to facilitate installation of an instrumented probe into a media by sonic drilling.

27. The apparatus of claim 8, wherein the insertion tube is of adequate durability to facilitate installation of an instrumented probe into a media by a combination of direct push and sonic drilling.

28. An apparatus which facilitates placing an instrumented probe into a media, comprising:
a plurality of stainless steel probe casings having first and second ends, the first end of one probe casing being configured to selectively couple with the second end of another probe casing at a casing joint to form an insertion tube, wherein the casing joint includes two seals which function as a substantial barrier to contaminants.

29. An apparatus which facilitates placing an instrumented probe into a media, comprising:
a plurality of probe casings having male and female threaded ends, the male threaded end of one probe casing being configured to selectively couple with the female threaded end of another probe casing at a casing joint to form an insertion tube, wherein the casing joint includes a seal which functions as a substantial barrier to contaminants, and wherein selectively coupling the male threaded end of one probe casing with the female threaded end of another probe casing requires about two and one-half turns to fully engage the casing joint and the seal.

30. An apparatus which facilitates placing an instrumented probe into a media, comprising:
a plurality of probe casings having male and female threaded ends, the male threaded end of one probe casing being configured to selectively couple with the female threaded end of another probe casing at a casing joint to form an insertion tube, wherein the casing joint includes two seals which functions as a substantial barrier to contaminants, and wherein selectively coupling the male threaded end of one probe casing with the female threaded end of another probe casing requires about two and one-half turns to fully engage the casing joint and the two seals, and wherein the probe casings have an outer wall defining an outside diameter, and wherein the outside diameter is about two and one-half inches.

31. A method of forming an insertion tube for placement of an instrumented probe into a media, comprising:
providing a plurality of probe casings, each probe casing having male and female threaded ends, the male threaded end of a first probe casing being configured to selectively couple with the female threaded end of a second probe casing at a casing joint to form an insertion tube;
providing at least one seal at the casing joint where the male threaded end of the c first probe casing and the female threaded end of the second probe casing are to be selectively coupled; and
turning the first and second probe casings relative to each other to selectively couple the male threaded end of the first probe casing with the female threaded end of the second probe casing to form the insertion tube.

32. The method of claim 31, and wherein the turning the first and second probe casings relative to each other to selectively couple the male threaded end of the first probe casing with the female threaded end of the second probe casing to form the insertion tube, comprises turning less than four turns to fully engage the casing joint with at least one seal.

33. The method of claim 31, wherein the turning the first and second probe casings relative to each other to selectively couple the male threaded end of the first probe casing with the female threaded end of the second probe casing to form the insertion tube, comprises about two and one-half turns to fully engage the casing joint and at least one seal.

34. The method of claim 31, and further comprising using the probe casings to install an instrumented probe into a media by direct push.

35. The method of claim 31, and further comprising using the probe casings to install an instrumented probe into a media by sonic drilling.

36. The method of claim 31, and further comprising using the probe casings to install an instrumented probe into a media by direct push and sonic drilling.

37. The method of claim 31, and further comprising using the probe casings to advance an instrumented probe into a media to a desired depth.

38. The method of claim 31, and further comprising using the probe casings to advance an instrument into a media to a depth greater than 30 meters.

39. The method of claim 37, and further comprising retrieving and reusing the probe casings.

40. The method of claim 37 and further comprising pressure testing the probe casings while the probe casings are in the media after advancing an instrument into the media.

41. The method of claim 31 wherein coupling the male threaded end of the first probe casing with the female threaded end of the second probe casing does not result in friction welding of the first probe casing to the second probe casing.

42. The method of claim 31 wherein coupling the male threaded end of the first probe casing with the female threaded end of the second probe casing does not result in galling of the first probe casing to the second probe casing.
